(19) United States
(12) Patent Application Publication
(10) Pub. No.: US 2010/0203124 A1
(43) Pub. Date: Aug. 12, 2010

(54) Kappa-2 Carrageenan Composition and Products Made Therefrom

(75) Inventors: James J. Modliszewski, Brick, NJ (US); Robert Kopesky, Camden, ME (US); Christopher J. Sewall, Hope, ME (US)

Correspondence Address: PATENT ADMINISTRATOR;FMC CORPORATION, 1735 MARKET STREET, PHILADELPHIA, PA, 19103, US

(73) Assignee: FMC CORPORATION, Philadelphia, PA (US)

(21) Appl. No.: 12/755,910

(22) Filed: Apr. 7, 2010

Related U.S. Application Data
(63) Continuation of application No. 11/810,661, filed on Jun. 6, 2007.
(60) Provisional application No. 60/813,205, filed on Jun. 13, 2006.
(60) Provisional application No. 60/811,160, filed on Jun. 6, 2006.

(51) Int. Cl. A61K 9/48; A61K 9/50; A61P 43/00; A23L 1/0532; A23L 1/05; C09D 105/00

(52) U.S. Cl. 424/456; 426/575; 426/89; 426/96; 426/573; 106/162.1

(57) ABSTRACT

The present invention is directed to a kappa-2 carrageenan composition comprising: (i) kappa-2 carrageenan, (ii) at least 70% sodium by weight of all cations in the composition; and (iii) free salt present in an amount of 0-25% by weight of the composition; wherein the composition has a viscosity of 20 cps to 40 cps. The present invention is also directed to products made from such kappa-2 carrageenan composition, as well as to processes of manufacture and use thereof.

This application claims the benefit of U.S. Provisional Application No. 60/813,205, filed Jun. 13, 2006, and U.S. Provisional Application No. 60/811,160, filed Jun. 6, 2006.

### FIELD OF THE INVENTION

The present invention is directed to a kappa-2 carrageenan composition comprising: (i) kappa-2 carrageenan, (ii) at least 70% sodium by weight of all cations in the composition; and (iii) free salt present in an amount of 0-25% by weight of the composition; wherein the composition has a viscosity of 20 cps to 40 cps. The present invention is also directed to products made from such kappa-2 carrageenan composition, as well as to processes of manufacture and use thereof.

### BACKGROUND OF THE INVENTION

Gelatin has long been used to form films useful in the preparation of soft capsules. It is a hydrolyzed protein from collagen usually obtained by boiling animal bones and cartilage under pressure with water. However, the use of gelatin suffers from several commercial drawbacks; e.g., its animal origins often preclude its availability to those who cannot or will not take animal derived capsules and recent concerns over bovine spongiform encephalopathy, BSE, or “Mad Cow Disease.”

As a result, academia and industry have been trying for many years to develop alternatives to gelatin that can desirably use many of the machines and processes, such as rotary dies, that are already in place to make soft capsules from gelatin alternatives.

For example, Japanese Patent Application Kokai Publication No. 61-10508A discloses capsules made from the composition of polysaccharides including at least carrageenan and polyhydric alcohols. Carrageenan can be used wholly or partly with other polysaccharides such as tamarind gum, pectin, gelatin, alginates, agar, furcellaran, cellulose derivatives, locust bean gum, and guar gum. Polyhydric alcohols include sorbitol, glucose, sucrose, ethylene glycol, diethylene glycol, triethylene glycol, polyethylene glycol, propylene glycol, polypropylene glycol, butane diol and glycerin. The soft capsules are made from concave stamping dies.

Japanese Patent Application Kokai Publication No. 63-164858 discloses mixtures of polysaccharides and polyhydric alcohols with/without alkaline substances. The broad list of polysaccharides purported to be useful in the application include natural polysaccharides such as carrageenan, alginic acid, alginate derivatives, agar, locust bean gum, guar gum, tamarind seed polysaccharides, pectin, xanthan gum, glucomannan, chitin, pullulan and cyclodextrine. The polysaccharides are stated to be combined with a concentrated water solution of at least one of a polyhydric alcohol, sugar alcohol, monosaccharide, disaccharide and oligosaccharide. The mixtures are stated to be useful in forming hulls of soft capsules. The three examples are directed to making hulls of soft capsules having double layers of the mixture with gelatin and a single layer consisting of the mixture of the invention with gelatin. No specific carrageenans are mentioned.

U.S. Pat. No. 5,089,307 discloses heat-sealable edible films comprising at least a film layer containing a water-soluble polysaccharide as the principal component, a polyhydric alcohol and water. The films are stated to be useful for sealing and packaging materials for dried foods, oily foods and the like. The polysaccharides purported to be useful include alginic acid and its salts (such as sodium salt); furcellaran; carrageenan such as kappa-, iota- and lambda-carrageenans; agar; pectin such as high-methoxy and low-methoxy pectins; gums such as tamarind seed gum, xanthan gum, guar gum, tara seed gum, locust bean gum; pullulan; chitin derivatives such as chitosan; starch such as wheat, corn and potato starches; dextrin; edible water-soluble cellulose derivatives such as carboxymethylcellulose; and mixtures of the foregoing. The weight ratio of the polyhydric alcohol to polysaccharide is preferably used in an amount of 1:5 to 1:1, and the polysaccharide is present in an amount of not less than 50% of the total amount of active components. There is no disclosure that such films can be used in the manufacture of soft or hard capsules.

U.S. Pat. No. 6,331,205 discloses aqueous viscous compositions for making soft or hard capsules containing carrageenan, preferably, iota carrageenan as the single gelling agent. Iota-, lambda-, mu-, and nu-carrageenans are disclosed as the types of carrageenans that can be used in the invention, and such are stated to be extracted from a variety of different seaweed sources depending on the extraction method utilized. Plasticizers are disclosed such as those belonging to the polyoxyls class; e.g., glycerol, sorbitol, maltodextrins, dextrose, mannitol, xylitol, polyoxyethylene glycol 400 to 6000, natural glycerides and hemisynthetics and their derivatives, etc. Soft capsules are said to be obtained by an adaptation of the “Scherer” method. Films made from kappa carrageenans are said to have syneresis causing problems in the manufacturing of hard and soft capsules. There is no description of any specific iota carrageenans, kappa carrageenans, kappa-2 carrageenans, etc.

U.S. Pat. No. 6,214,376 discloses gelatin-free capsules made from compositions comprising water-soluble hydrophilic colloidal layers comprising gel films of kappa-carrageenan and a plasticizer. The gelatin free soft capsules are said to be made from kappa-carrageenan as the main gel-forming polymer (at least 50% by weight of gums that form thermoreversible gels or contribute to the formation of thermoreversible gels). Hydrolyzed starches such as maltodextrin may be added to increase solids concentration, aid heat sealing and prevent hazing induced by gelling salts. Other types of gums, such as iota carrageenan, are taught to be minimized, most preferably, to an amount less than 0.5% of the total film composition.

U.S. Pat. No. 6,340,473 requires the use of a modified starch having a hydration temperature below about 90° C. and iota carrageenan for the manufacture of soft capsules using rotary die encapsulation apparatus. The weight ratio of the modified starch to the iota carrageenan is stated to be crucial to forming a satisfactory film. That is, the weight ratio of the modified starch to the iota carrageenan is said to be 1.5:1. The inventors purportedly found that iota-carrageenan alone does not produce an acceptable film and that modified starch alone does not produce an acceptable film useable for encapsulation. The stated theory is that the iota carrageenan functions as an elasticizing agent rendering an otherwise inelastic, modified starch film, elastic. Carrageenans are stated to be complex with hundreds of different products on the market having different functionalities. Eucheuma spinosum is stated to be the seaweed source for iota carrageenan, and not all carrageenans are stated to be useable in the invention, e.g., kappa carrageenan is stated not to be a substitute for iota carrageenan therein.

It is known that certain high solids, low moisture film forming compositions containing, for example, hydrocolloids, form highly viscous solutions that make formation of hydrated films difficult to obtain.

In addition, many attempts have been made to make soft capsules from high solids, low moisture films such as hydrocolloids. However, such attempts to make soft capsules have suffered from the drawback mentioned above. That is, hydrocolloids are known to form highly viscous solutions that are difficult to sufficiently hydrate and form a film in conventional soft capsule making processes.

US 2005/0019374 and US 20050014852 disclose the use of kappa-2 carrageenans in general as well as very low molecular weight carrageenans, including kappa-2 carrageenans, in making gel films and soft capsules

### SUMMARY OF THE INVENTION

The present invention is directed to a kappa-2 carrageenan composition comprising: (i) kappa-2 carrageenan, (ii) at least 70% sodium by weight of all cations in the composition; and (iii) free salt present in an amount of 0-25% by weight of the composition; wherein the composition has a viscosity of 20 cps to 40 cps. The present invention is also directed to products made from such kappa-2 carrageenan composition, as well as to processes of manufacture and use thereof.

### DETAILED DESCRIPTION OF THE INVENTION

Kappa-2 carrageenan is generally contained in a composition containing cations complexed therewith, as well as other cations and/or anions that are free salts not complexed with the kappa-2 carrageenan. The kappa-2 carrageenan composition of the present invention comprises kappa-2 carrageenan and at least 70% sodium cation by weight of the cations in the composition (free or complexed with the kappa-2 carrageenan), more particularly, at least 75%, at least 80%, at least 85%, at least 90%, at least 92%, at least 95%, at least 98% sodium cation, all based on the total weight of the cations in the composition. The composition further contains free salts in an amount of 0% to 25% based on the total weight of the composition, more particularly, 0 to 20%, 0 to 15%, 0 to 10%, 0 to 8%, 0 to 5%, and 0 to 2%, all based on the total weight of the composition. In other embodiments of the invention, the composition contains free salts in the amount of 0.001% to 20%, 0.001% to 15%, 0.001% to 10%, 0.001% to 8%, 0.001% to 5%, or 0.001% to 2%, all based on the total weight of the composition.

The amount of free salt in the composition is determined by washing the kappa-2 carrageenan composition in a mixture of isopropanol and water (60% isopropanol) for two hours, separating the kappa-2 carrageenan from the water and alcohol mixture by filtration, separately drying the kappa-2 carrageenan and the water and alcohol mixture (filtrate), and calculating the percent recovered carrageenan and free salt (from the dried filtrate), correcting for the moisture content in the kappa-2 carrageenan. The kappa-2 carrageenan composition of the present invention also has a viscosity of 20 cps to 40 cps, more particularly, from 20 cps to 36 cps, when measured using a Brookfield LV viscometer with appropriate spindles and speeds in a 1.5% solids in water solution at 75° C. The tested solution does not contain any added salts.

Further, kappa-2 carrageenan compositions consisting of, or consisting essentially of, (i) kappa-2 carrageenan, (ii) at least 70% sodium by weight of all cations in the composition; and (iii) free salt present in an amount of 0-25% by weight of the composition; wherein the composition has a viscosity of 20 cps to 40 cps, more particularly, from 20 cps to 36 cps, are also included within the scope of the present invention.

Without being bound by any theory, it is believed that the kappa-2 carrageenan compositions of the present invention (containing the amount of sodium and free salt as well as having the viscosity of 20 cps to 40 cps) surprisingly enable the manufacture of gel films having a high solids system that are strong and elastic.

Carrageenan is a commercially significant galactan polysaccharide found in red seaweed. All carrageenans contain repeating galactose units joined by alternating α1→3 and β1→4 glycosidic linkages and are sulfated to widely varying degrees. The types of carrageenan may be distinguished, in part, by their degree and position of sulphation, as well as the seaweed from which they are obtained. For example, iota carrageenan has a repeating unit of D-galactose-4-sulfate-3,6-anhydro-D-galactose-2-sulfate providing a sulfate ester content of about 25 to 34%. Iota carrageenan can be obtained, for example, from Eucheuma denticulatum (“also referred to as “Spinosum”). Kappa carrageenan has a repeating unit of D-galactose-4-sulfate-3,6-anhydro-D-galactose and is obtained, for example, from Kappaphycus alvarezii (also known as “Eucheuma cottonii”). In contrast, kappa-2 carrageenan is reported by R. Falshaw, H. J. Bixler and K. Johndro, Structure and Performance of Commercial Kappa-2 Carrageenan Extracts, Food Hydrocolloids 15 (2001) 441-452, and by H. Bixler, K Johndro and R Falshaw, Kappa-2 carrageenan: structure and performance of commercial extracts II, Food Hydrocolloids 15 (2001) 619-630 to be copolymers containing a certain amount of kappa repeating units (3:6-anhydrogalactose (3:6-AG)) and iota repeating units (3:6-anhydrogalactose-2-sulfate (3:6-AG-2-S)) covalently bound in the copolymer backbone and obtained from certain Gigartinaceae algae. The foregoing references state that such kappa-2 carrageenans have distinctly different properties as compared to simple mixtures of kappa and iota carrageenans. Other references discussing kappa-2 carrageenan are discussed in these publications. Kappa-2 carrageenan extracted from Gigartina atropurpurea is reported by R. Falshaw, H Bixler and K Johndro, Structure and Performance of Commercial Kappa-2 Carrageenan extracts III, Food Hydrocolloids 17 (2003) 129-139. While there has been considerable confusion historically about the physical nature of kappa-2 carrageenans, recent studies, such as those mentioned immediately above, have confirmed that kappa-2 carrageenans are copolymers containing kappa and iota repeating units covalently bound (in certain ratios of kappa to iota moieties) in clear distinction to physical mixtures of kappa and iota polymers.

As used herein, kappa-2 carrageenan has a molar ratio of 3:6 AG-2S to 3:6 AG content of 25 to 50%, iota carrageenan has a molar ratio of 3:6 AG-2S to 3:6 AG content of 80 to 100% and kappa carrageenan has a molar ratio of 3:6 AG-2S to 3:6 AG content less than that for kappa-2 carrageenan. For example, kappa carrageenan from Eucheuma cottonii, a commonly known and used seaweed source for kappa carrageenan, has a molar ratio of 3:6 AG2S to 3:6 AG content of less than about 10%; and iota carrageenan from Spinosum, a commonly known and used seaweed source for iota carrageenan, has a molar ratio of 3:6 AG2S to 3:6 AG content greater than about 85%. This means that kappa-2 carrageenan comprises a ratio of kappa (3:6-AG) repeating units to iota (3:6-AG-2-S) repeating units between 1.0 to 3.0:1, more particularly, 1.5 to 3.0:1 (more particularly depending on the desired application). The molar ratio of 3:6 AG-2S to 3:6 AG content of 25 to 50% holds in kappa-2 carrageenans regardless of its degree of modification and precursor content (e.g, mu and nu repeating units). Thus, any kappa-2 carrageenan meeting the molar ratio of 3:6 AG-2S to 3:6 AG content of 25 to 50%, regardless of its degree of modification, is within the scope of this invention.

The kappa-2 carrageenan to be used in the present invention may be contained within or purified or separated from a number of seaweed species within the class of, for example, Gigartinaceae algae such as Gigartina radula, Gigartina corymbifera, Gigartina skottsbergii, Iridaea cordata, Sarcothalia crispata, and Mazzaella laminarioides. The seaweed source of the kappa-2 carrageenan to be used in this invention is any that produces kappa-2 carrageenan having the molar content of 3:6 AG-2S to 3:6 AG described herein. The kappa-2 carrageenan used in the present invention can be obtained from a single seaweed source, or from a mixture of two or more different seaweed sources. The kappa-2 carrageenan that can be used in the present invention may occur naturally in the seaweeds above or may be modified from the above seaweeds to increase the amount of 3:6 AG-2S and 3:6 AG moieties in the kappa-2 carrageenan from their precursors (e.g., 3:6 AG-2S moiety within the kappa-2 carrageenan modified from its precursor nu upon alkali treatment, and 3:6 AG moiety within the kappa-2 carrageenan modified from its precursor mu upon alkali treatment). The recovery and modification techniques are well known in the art including the cited publications by Falshaw, Bixler and Johndro. For example, modification of the kappa-2 carrageenan can occur during its recovery from certain Gigartinacean algae as a result of alkali treatment at elevated temperatures. Recovery methods include the optional full or partial filtration of insolubles from the starting material or the use of unfiltered material. When the nu and mu precursors in the kappa-2 carrageenan are modified to 3:6 AG-2S to 3:6 AG, respectively, such modification may be complete (i.e., 100% of the nu and mu precursors in the kappa-2 carrageenan are modified to 3:6 AG-2S and 3:6 AG moieties, respectively) or less than fully complete (i.e., less than 100% of the nu and mu precursors in the kappa-2 carrageenan are modified to 3:6 AG-2S and 3:6 AG moieties, respectively). It is understood that during the recovery process of the kappa-2 carrageenan from the above seaweeds small or trace amounts of other carrageenans may be present (e.g., lambda carrageenans) and such can be used with the kappa-2 carrageenans in the present invention.

The kappa-2 carrageenan of the present invention can be prepared using any conventional process, for example, processes involving: washing the seaweed to remove extraneous material, separating the lambda fraction of the seaweed (e.g., manually), bleaching to reduce color, washing in KCl or KCl and combinations of sodium chloride to remove excess bleach, color bodies, etc., modifying the seaweed in alkali and KCl or combinations of KCl and NaCl, washing in KCl or combinations of KCl and NaCl to remove excess alkali and non-carrageenan seaweeds, neutralizing in KCl or combinations of KCl and NaCl to approximately neutral pH, a second bleaching step, viscosity adjustment, additional washing steps in NaCl, drying, grinding, solubilizing in hot water and NaCl to further ion exchange the carrageenan, viscosity adjustment with H2O2, filtration, concentration, precipitation with isopropyl alcohol, dewatering, washing in isopropyl alcohol, drying and grinding. Conventional ion exchange processes can be utilized to make the kappa-2 carrageenan compositions of the present invention, e.g., such conventional processes as dialysis; washing with salt, water and alcohol; and diafiltration (using a membrane).

The products that can be made from the kappa-2 carrageenan composition of the present invention include homogeneous gel films, delivery systems, barriers, controlled release systems, oral dose forms such as hard capsules, soft capsules, enrobed solid materials (such as powders, aggregates), food products, agricultural products, industrial products such as paintballs, etc. The kappa-2 carrageenan can also be used in coatings for a variety of substrates and such coatings can be applied in multiple layers to any such substrate.

The kappa-2 carrageenan composition can be used to make gel films by using the kappa-2 carrageenan in a film forming amount (i.e., an amount that adds film strength to the gel film) which is distinguished from trace amounts of kappa-2 carrageenan that do not add film properties to the film. Thus, for example, in a gel film of the present invention containing the second film formers discussed below, a film forming amount of kappa-2 carrageenan is an amount that adds film strength to the overall film. Such film forming amounts are generally at least 0.5% by weight of the gel film, particularly, 0.5% to 90%, more particularly, 0.5% to 50%, more particularly, 0.5% to 25%, more particularly, 1.5% by weight of the dry gel film depending on the application.

As used herein, “homogeneous film” defines films that, to the naked eye, are visually uniform and free of defects such as lumps, cracks, particles of the primary structure forming components that are undissolved that should be dissolved, non-uniform distribution of insoluble particles, etc. “Fish eyes” (mixed liquid and solid states) or “gel balls” (non-uniform gel structure) would not meet the definition of “homogeneous” as used herein.

The gel films of the present invention are homogeneous, thermoreversible gel films. They can be cast and used in a wide variety of applications as cast films or in subsequent processing.

As used herein, “thermoreversible film” defines a film that has a melting temperature. As used herein, the melting temperature is the temperature or temperature range over which the gel film softens or flows due to gravitational or induced forces to move the molten mass.

As used herein, the phrase “gel films” refer to a thin membrane or three-dimensional network (sponge-like), formed from structured kappa-2 carrageenan. The gel-forming composition is characterized by a gel temperature, the temperature below which the molten mass of the gel composition must be cooled to form a self-supporting structure. Optionally, a molten mass can be cast hot and allowed to cool, as well as dry to further concentrate the solids (controlled moisture removal) until a gel film is formed by the gel composition. The melt temperature of a thermoreversible gel film is higher than its gel temperature.

The homogeneous, thermoreversible gel film of the present invention can optionally contain at least one of a plasticizer, a second film former, a bulking agent and a pH controlling agent. The components to be added to the gel film and their amounts can vary depending on the desired use of the kappa-2 gel film.

Examples of such a plasticizer include polyols such as glycerin, sorbitol, maltitol, lactitol, corn starch, fructose, polydextrose and polyalkylene glycols such as propylene glycol and polyethylene glycol. The amount of the plasticizer can vary depending on the use of the gel film and its desired elasticity. For example, such plasticizers can generally be used in an amount of at least 5%, more preferably, at least 10%, more preferably, at least 20%, more preferably, at least 30% by weight of all the components including water in the dry film if a gel film having more elasticity is desired; e.g., films to be used to make soft capsules. For other applications, such as hard capsules, where less elastic films are desired, the plasticizer can be present in an amount of 0% to 20% by weight of all the components in the dry film. It is possible that the gel film of the invention contains no plasticizer at all.

Examples of the second film former that can be used in the present invention include at least one of a starch, starch hydrozylate, starch derivative, digestion resistant maltodextrins, cellulose gum, hydrocolloid, an alkylcellulose ether or a modified alkyl cellulose ether. Examples of the hydrocolloid include at least one of kappa carrageenan; iota carrageenan; kappa and iota carrageenans having a reduced molecular weight (e.g., having an extract viscosity of 20 cps or less at 75° C. in a 1.5% aqueous sodium chloride solution); alginates including potassium alginate, sodium alginate, ammonium alginate and propylene glycol alginate; polymannan gums (e.g., generally less than about 1000 mPs viscosity as measured at 1 wt % in water at 25° C.) such as low viscosity guar gum; pullulan, gellan (including high and low-acyl gellan); pectin and less than fully modified versions thereof and combinations thereof. An example of an alkylcellulose ether that can be used in the present invention is hydroxyethylcellulose. Examples of modified alkylcellulose ethers that can be used in the present invention include hydroxypropylcellulose and hydroxypropylmethylcellulose. The kappa-2 carrageenan can be the only film former in the gel film. When the gel films of the present invention contain second film formers, the kappa-2 carrageenan can be present in an amount of at least 10%, at least 20%, at least 50% or at least 80% by weight of the total amount of film formers in the dry gel film. A dried film is the controlled residual form of a cast film, as described within this application. Combinations of ingredients, such as: kappa-2 carrageenan, and, optionally, a starch, a polyol and water for processing, are dispersed, hydrated, solubilized and, optionally, de-aerated within the process options described within. The resulting homogeneous mass is cast or formed at the desired solids level (necessary to achieve the intended end-product). The cast system is formed, via gravitational or controlled forces, and subsequently either immediately further processed (such as soft gel capsule production) or the cast mass is additionally processed by utilizing various methods for uniform and controlled water removal until the desired moisture level is reached. Controlled water removal from the cast system allows a further strengthening/alignment of the homogeneous film ingredients into a denser structure, which can further strengthen film characteristics. Moisture removal is limited to that moisture not bound to the molecular surface of the various hydrocolloid and carbohydrate components. The dried film is achieved when the originally cast film does not lose additional weight while subject to the various drying methods employed in the dewatering/dehydration process. A reduction in moisture content to constant levels also imparts stability to the film and, optionally, its contents (if embedded or enrobed or entrapped, etc.) as water activity is also reduced by the process. It is understood that the resulting cast film can be fully dried or to an intermediate, retained moisture level between the cast film moisture level and the maximum dried moisture level, depending upon the final product use and functions.

Examples of the bulking agent include non-colloidal (vegetal sourced) cellulose, microcrystalline (vegetal sourced) cellulose, microcrystalline starch, modified and unmodified starch, starch derivatives and fractions, inulin, digestion resistant maltodextrins, starch hydrozylates, sugar, corn syrup and polydextrose. As used herein and in the claims, the term “modified starch” includes such starches as hydroxypropylated starches, acid-thinned starches, and the like. Examples of modified starches that can be used in the present invention include Pure Cote™ B760, B790, B793, B795, M250 and M180, Pure-Dent™ B890 and Pure-Set™ B965, all available from Grain Processing Corporation of Muscatine, Iowa, and C AraTex™ 75701, available from Cerestar, Inc. Examples of starch hydrozylates include maltodextrin also known as dextrin. An example of a digestion resistant maltodextrin that can be used in this invention includes Fibersol-2 available from Archer Daniels Midland/Mitsutani America. Unmodified starches such as potato starch can also contribute to the film strength when combined with the hydrocolloids within the scope of the invention. In general, modified starches are products prepared by the chemical treatment of starches, for example, acid treatment starches, enzyme treatment starches, oxidized starches, cross-bonding starches, and other starch derivatives. It is preferred that the modified starches be derivatized wherein side chains are modified with hydrophilic or hydrophobic groups to thereby form a more complicated structure with a strong interaction between side chains.

The amount of the bulking agent to be used in the present invention is generally in the amount of 0 to 20% by weight of the dry film, but more can be used, if desired, for example, at least 20%, more preferably, at least 30% by weight of the dry film.

Note that starch, starch derivatives, digestion resistant maltodextrin and starch hydrozylates can be multifunctional. That is, in addition to being used as bulking agents, they can be used as second film formers. When such are used as bulking agents and second film formers, they are generally used in an amount of at least 10%, preferably, at least 20%, more preferably, at least 30% by weight of the dry gel film depending on the application; e.g., soft capsules.

Examples of the pH controlling agent that can optionally be used in the present invention include bases such as hydroxides, carbonates, citrates and phosphates, mixtures thereof and their salts (e.g., sodium citrate). The pH controlling agent can be chosen as the source of added beneficial cations such as potassium or sodium. For some compositions, the pH controlling agent can be used to improve the stability of the gel film. The amount of the pH controlling agent is generally in the amount of 0 to 4%, preferably, 0 to 2%.

The gel films of the invention can also contain colorants and/or flavorants such as sugar, corn syrup, fructose, sucrose, etc, and/or antioxidants, such as anthocyanins, whether or not other components, such as plasticizers, bulking agents, second film formers, etc. are present. One embodiment of a gel film of the invention comprises kappa-2 carrageenan, flavorant and water in a high solids system; e.g., greater than 50%, 60%, 65%, 75%, 80%, 85%, 90° A solids.

The dry gel films (e.g., 80% solids or higher) of the present invention have been found to have, for example, a break force of at least 2,500 grams, at least 4,000 grams, at least 5,000 grams and at least 6,000 grams, as determined using a Texture Analyzer TA-108S Mini Film Test Rig. At lower solids, the gel films have been found to have a break force of at least 50 grams, at least 100 grams, at least 200 grams, at least 500 grams, at least 1000 grams, as determined in a similar manner.

The films of the present invention have been found to have a solids content of at least 50%, at least 60%, at least 70%, at least 80% and at least 90% of all components in the gel film. It is understood that 15%, 10% or 5% water may remain strongly associated with the solids in the gel film.

Dry film thicknesses generally used for soft capsules are in the range of 0.5 to 3.0 mm, more preferably, 0.8 to 1.2 mm.

It is possible that the gel films of the present invention can contain nonthermoreversible gums. However, so as not to adversely impact the homogeneous and thermoreversible nature of the gel films of the present invention, such nonthermoreversible gums should be present in an amount of less than 50% by weight of the kappa-2 carrageenan, preferably, less than 40%, more preferably, less than 30%. Examples of such nonthermoreversible gums include crosslinked (and partially crosslinked) gums such as calcium set (e.g., crosslinked) pectins and/or alginates. Calcium reactive alginates and pectins, as well as their less refined forms, are considered as thermoreversible gums in the absence of divalent cations. Other non-thermoreversible gums such as tragacanth gum contribute to the thermoreversability of the kappa-2 carrageenan by absorption of water within its structure thereby causing the kappa-2 carrageenan to form a denser, three-dimensional structure, as it is solubilized in less water, providing the same effect as increasing the kappa-2 carrageenan amount without the secondary film formers. Additional film formers, such as polymannans can form continuous networks, either by themselves or synergistically with other components during the activation and casting process. These polymannans can be optionally used in various molecular weights (related to their chain length/size) which allow their use without substantially affecting the process of the molten mass and film formation.

The kappa-2 carrageenan gel films of the present invention are generally made from a process utilizing an apparatus that enables sufficiently high shear, temperature (above the gelling temperature) vacuum control and residence time so as to provide a homogeneous molten mass of the composition and formation of the gel upon cooling. Such apparatus include but are not limited to Ross mixers, Stephan processors, conventional jet cookers and extruders. Ross mixers, Stephan processors, extruders and conventional jet cookers are readily available commercially. Prior to cooling, the molten mass can also be fed to at least one of a pump, mixer or devolatilizer. An example of a device that performs any one of such functions is an extruder. An extruded molten mass can also be directed to a film forming or shaping device (e.g. open or closed spreader box or die, as used in a capsule forming machine) that aids in the uniform casting of a continuous film, or, through a die that allows a direct formation of a film from the molten mass delivery equipment. Care must be taken to maintain the molten mass above the initiation of restricted flow/gel structure formation. Insulated and pre-heated (to maintain proper temperatures) transfer hoses may be used to insure molten mass flow until desired gel film formation is initiated on the casting rolls or at other film formation points, such as an extruder (restrictive flow, film forming device) or die. Additional processing methods (such as pre-heating the discharge/plunger-like head as seen in a Ross process system) can force (by pressure) the molten mass through the transfer hoses mentioned above. Additional insulation can help maintain molten mass temperatures, for example, through the use of a Teflon disk initially placed upon the molten mass surface immediately after removing the mixing device. In addition, the feeder hoses can be introduced to the heat controlled molten mass feeder (casting) boxes located on a capsule machine either directly to the boxes or through an optional modification of the feeder boxes which introduces a top half enclosure/cover that helps maintain molten mass temperatures within the feeder box, reduces moisture loss, and maintains uniform (center) filling of the box during the extended process of forming films for capsules. It is understood that other methods of maintaining molten mass temperatures can be used to form films for capsules. This includes, but is not limited to: extrusion of the molten mass through dies/orifices into films that: can be immediately fed into the capsule forming apparatus, stored at temperatures that maintain proper film conditions (to form capsules) until needed, or dried to desired moisture, solids and texture levels, until needed. Such dried films have the property of re-absorbing water (water is introduced by any means) throughout its gel film matrix. Moisture is introduced to the film until a desired moisture content and strength/texture is reached that will allow the film's introduction into a capsule machine to make soft capsules. It is also understood that the homogeneous molten mass can be transferred to a heated reservoir or holding tank until the mass is further transferred (at a sufficient temperature to all its redirection by the use of pumps through heated hoses to the area where the molten mass is cast, formed, cooled to gel, and further utilized or processed for the cited applications).

The process for making soft capsules from the kappa-2 carrageenan gel films of the invention includes the use of any conventional encapsulating apparatus, e.g., a conventional rotary die apparatus or concave stamping die. For example, once the molten mass of the present invention has been made, it can be cast onto drums, cooled and then fed between rotary encapsulation dies where the films are heated again, filled, sealed and cut. For a good description of this conventional process, see WO 98/42294. Alternatively, and as benefit of the present invention over conventional soft capsule processes, the use of the high shear apparatus disclosed above allows the molten mass to be sufficiently hydrated, applied to drums as they are cooling and then fed into conventional encapsulating apparatus for filling, sealing, and cutting. This continuous type process can be used to eliminate the step of having to reheat fully gelled and cooled films. The above rotary die process can be used to make soft capsules of the invention having any desired shape.

The fill materials for the soft capsules can be any materials widely used in the above rotary die process, including pharmaceutical ingredients, agricultural ingredients, nutraceutical ingredients, veterinary ingredients, foods, cosmetics, personal care, industrial, etc. and can be a liquid, solid, suspension, dispersion, etc.

The present invention is also directed to a solid form comprising a fill material encapsulated by the homogeneous, thermoreversible gel film of the present invention. One type of such solid form is a hard capsule. Hard capsules, as used herein, refer to those solid forms that are conventionally used, e.g., in the pharmaceutical industry whereby two half shells are formed, a fill material, usually a powder, is placed in the shells and the two halves are placed together to form the hard capsule. One process for making such hard capsules would typically involve dipping metal pins or bars into the molten composition of the present invention and allowing the gel film to form around the pins. The gel films are dried and then removed from the pins. These processes are well known in the industry as methods of making hard capsules. The fill materials for the hard capsules can be any fill materials commonly used in such dosage forms. Generally, the fill materials can be liquids (including emulsions) or solids such as powders. The fill materials can be a pharmaceutical ingredient, agricultural ingredient, nutraceutical ingredient, veterinary ingredient, food, cosmetic ingredient, etc.

The solid form may also encapsulate a powder, tablet, caplet, microcapsule or capsule in accordance with known techniques. For example, encapsulating a hard capsule with the gel film of the invention would allow for safety seal/tamper resistant capabilities.

The gel film can also be used to modify the dissolution profile of the dosage forms. For example, gel films of the invention can contain added components that can create solid dosage forms having immediate release, controlled, enteric or delayed release capabilities or can be released upon activation by a known event, condition or process. Definitions of “immediate release”, “delayed release” and “enteric” can be found in the U.S. Pharmacopeia and are incorporated herein by reference.

The present invention is now described in more detail by reference to the following examples, but it should be understood that the invention is not construed as being limited thereto. Unless otherwise indicated herein, all parts, percents, ratios and the like are by weight.

### EXAMPLES

The following are prepared examples of the kappa-2 carrageenan compositions of the present invention.

TABLE

| | A | B | C | D |
|---|---|---|---|---|
| | wt % of | wt % of | wt % of | wt % of |
| Sample | composition | composition | composition | composition |
| Chemical | | | | |
| Analysis | | | | |
| Cl− | 0.11 | 0.14 | 0.10 | 0.14 |
| K+ | 1.09 | 0.94 | 1.05 | 1.09 |
| Na+ | 5.38 | 5.16 | 5.26 | 5.41 |
| Ca++ | 0.17 | 0.19 | 0.20 | 0.24 |
| Mg++ | 0.01 | 0.01 | 0.01 | 0.01 |
| Gum | 88.76 | 88.3 | 91.3 | 91.0 |
| Content | | | | |
| Physical | | | | |
| Analysis | | | | |
| Moisture | 12.1 | 13 | 10.3 | 9.5 |
| Content | | | | |
| Viscosity | 33 | 36 | 33 | 32 |
| (cps) | | | | |
| pH | 8.8 | 9.5 | 9.5 | 9.4 |

Properties were determined for the samples and are reported in the Table. The gum recovered as the gum content is kappa-2 carrageenan. The gum content is determined by the following process: mixing a 1 gram sample of the kappa-2 carrageenan composition with 200 ml of an alcohol/EDTA solution containing 5 grams of Na₄EDTA (ethylene-diamine tretraacetic acid) in 1,000 ml of 60% isopropanol (a mixture of 60% isopropanol and 40% water by weight), and stirring for two hours; filtering the sample, and washing the sample with successive additions (two washes of about 50 ml each) of 65% isopropanol (a mixture of 65% isopropanol and 35% water by weight) to remove Na₄EDTA. The gum remaining after washing the sample is dried in an oven at 60° C. for at least thirty minutes, and then in a vacuum oven at 70° C. overnight, and cooled to room temperature in a desiccator. The recovered gum is weighed and the gum content is calculated as the percent recovered. The weight % provided in the table above is the weight percent based on the total weight of all components in the kappa-2 composition. Cation concentration was determined by atomic absorption (AA) spectroscopy. Chloride concentration was determined by titration. Moisture content was determined by drying overnight at 70° C. in a vacuum oven. Viscosity of the kappa-2 carrageenan compositions was determined at 75° C. for a 1.5% aqueous solution using a Brookfield LVF viscometer with spindle #1 at 30 rpm.

While the invention has been described in detail and with reference to specific embodiments thereof, it will be apparent to one skilled in the art that various changes and modifications can be made therein without departing from the spirit and scope thereof.

What is claimed is:

1. A kappa-2 carrageenan composition comprising: (i) kappa-2 carrageenan, (ii) at least 70% sodium by weight of all cations in said composition; and (iii) free salt present in an amount of 0-25% by weight of the composition; wherein said composition has a viscosity of 20 cps to 40 cps.

2. The kappa-2 carrageenan composition of claim 1 wherein said composition has a viscosity of 20 cps to 36 cps.

3. The kappa-2 carrageenan composition of claim 1, wherein said sodium cation is present in an amount of at least 75% by weight of the cations in said composition.

4. The kappa-2 carrageenan composition of claim 1, wherein said sodium cation is present in an amount of at least 80% by weight of the cations in said composition.

5. The kappa-2 carrageenan composition of claim 1, wherein said sodium cation is present in an amount of at least 85% by weight of the cations in said composition.

6. The kappa-2 carrageenan composition of claim 1, wherein said sodium cation is present in an amount of at least 90% by weight of the cations in said composition.

7. The kappa-2 carrageenan composition of claim 1, wherein said sodium cation is present in an amount of at least 92% by weight of the cations in said composition.

8. The kappa-2 carrageenan composition of claim 1, wherein said sodium cation is present in an amount of at least 95% by weight of the cations in said composition.

9. The kappa-2 carrageenan composition of claim 1, wherein said sodium cation is present in an amount of at least 98% by weight of the cations in said composition.

10. The kappa-2 carrageenan composition of claim 1, wherein said free salt is present in an amount of 0 to 20% by weight of said composition.

11. The kappa-2 carrageenan composition of claim 1, wherein said free salt is present in an amount of 0 to 15% by weight of said composition.

12. The kappa-2 carrageenan composition of claim 1, wherein said free salts is present in an amount of 0 to 10% by weight of said composition.

13. The kappa-2 carrageenan composition of claim 1, wherein said free salt is present in an amount of 0 to 8% by weight of said composition.

14. The kappa-2 carrageenan composition of claim 1, wherein said free salt is present in an amount of 0 to 5% by weight of said composition.

15. The kappa-2 carrageenan composition of claim 1, wherein said free salt is present in an amount of 0 to 2% by weight of said composition.

16. The kappa-2 carrageenan composition of claim 1, wherein said free salt comprises at least one of calcium, potassium, magnesium, sodium and chlorine.

17. A solid form comprising a fill material encapsulated by a homogeneous, thermoreversible gel film comprising the kappa-2 carrageenan composition in any of claims 1-16.

18. The solid form of claim 17, wherein said solid form is a soft capsule or hard capsule.

19. The solid form of claim 17, wherein said fill material comprises a powder, tablet, caplet, microcapsule or capsule.

20. A homogeneous, thermoreversible gel film comprising a film forming amount of the kappa-2 composition in any one of claims 1 to 16 and, optionally, at least one plasticizer, a second film former, a bulking agent, and a pH controlling agent.

21. A coating composition comprising the kappa-2 composition in any of claims 1 to 16.
